(19) United States
(12) Patent Application Publication
(10) Pub. No.: US 2021/0281475 A1
(43) Pub. Date: Sep. 9, 2021

(54) SYSTEMS AND METHODS FOR STEERING NETWORK DYNAMICS

(71) Applicants: Enrico Borriello, Tempe, AZ (US); Kimberly Bussey, Phoenix, AZ (US); Sara Imari Walker, Scottsdale, AZ (US)

(72) Inventors: Enrico Borriello, Tempe, AZ (US); Kimberly Bussey, Phoenix, AZ (US); Sara Imari Walker, Scottsdale, AZ (US)

(73) Assignee: Arizona Board of Regents on behalf of Arizona State University, Tempe, AZ (US)

(21) Appl. No.: 17/179,091

(22) Filed: Feb. 18, 2021

Related U.S. Application Data
(60) Provisional application No. 62/978,135, filed on Feb. 18, 2020.
(60) Provisional application No. 63/028,992, filed on May 22, 2020.

(51) Int. Cl. H04L 12/24; G06F 30/20; G06F 8/61

(52) U.S. Cl. CPC H04L 41/0806; G06F 8/61; G06F 30/20

(57) ABSTRACT

Various embodiments of a system and associated method for steering gene regulatory networks, among other network types, to a desired attractor state are disclosed herein. In one embodiment, the method involves identifying duplicative nodes and iteratively simulating effects of duplicated nodes on the network to steer the network to a target attractor state.

### CROSS REFERENCE TO RELATED APPLICATIONS

The present document is a Non-Provisional patent application that claims benefit to U.S. Provisional Patent Application Ser. No. 62/978,135 filed 18 Feb. 2020 and U.S. Provisional Patent Appln. 63/028,992 filed 22 May 2020, which are herein incorporated by reference in their entireties.

### FIELD

The present disclosure generally relates to complex network engineering; and in particular, to steering node expression in complex networks using node duplication.

### BACKGROUND

A critical problem in engineering of complex networks is the need to develop a capacity to steer complex network systems having a plurality of interconnected nodes to a specified target “attractor” state (i.e. a steady-state final configuration of varying statuses of each node which the network tends to converge to). Current methods attempt to steer these complex networks to the desired attractor state by continually intervening with the dynamics of the system. However, these methods are costly in terms of overhead and may require human operators. In addition, if intervention is terminated during the steering process, the network has no intrinsic capacity to regulate its own dynamics to converge to the target attractor state of interest.

Understanding the mechanisms underlying the emergence and persistence of new cell types is a central problem in the evolution and development of multicellular organisms. Whereas all cell types can in principle access the same genetic information, in practice, regulation of gene expression restricts this such that only a subset of an organism's total genomic information content is accessible to a given cell type at a given time, permitting differentiation of many phenotypes from a single genotype. Regulation of gene expression therefore plays a dominant role in establishing cell types. From a formal point of view, the question of how new cell types emerge, therefore, reduces the problem of understanding how new regulatory structures evolve that can specify and control the expression of novel phenotypes.

The interplay among these regulatory genes and their interaction with the other components of the cell governs the expression levels of both mRNA and proteins, where the set of interactions are described as a gene regulatory network (GRN). If multicellular phenotypes are the product of developmental differentiation processes controlled by GRNs, as the raw material for evolutionary change, every phenotypic variant is expected to be the product of a corresponding change in those regulatory networks.

In the case of evolution, continuous feedback in the form of selection or drift leads to changes in the underlying network architecture controlling individual phenotypes, for instance by means of mutation, gene duplication or deletion, etc.

In the case of developmental differentiation, time-dependent extracellular feedback over the regulatory network assures that the steady gene expression patterns follow a specific developmental sequence. Such extracellular signaling is not required to change the underlying structure of the GRN for steady activation pattern to change. Therefore, when a GRN is described with the language of dynamical systems, the question arises of whether cellular behavior can be controlled through the continued regulation of just a minimal set of nodes via external signals.

The evolutionary scale is the one posing the hardest problems in modeling the emergence of novel cell phenotypes, and the retainment of old ones. In fact, any attempt at trying to predict the steady states of the regulatory process—i.e. the cell types—in terms of a dynamical model (coupled ordinary differential equations, Boolean network models, stochastic gene networks, to name a few common approaches) faces the difficulty of having to reconcile the fixed number of genes in these models, whose expression level is representative of a given cell type, with the possibility for the size of the genotype to change over evolutionary timescales. Chromosome loss and gain, single gene and whole genome duplication, as well as horizontal gene transfer, all alter the number of genes participating in the dynamics of a GRN. In doing so, these processes deprive the mapping of cell types to gene expression patterns of its original meaning.

It is with these observations in mind, among others, that various aspects of the present disclosure were conceived and developed.

### BRIEF DESCRIPTION OF THE DRAWINGS

FIG. 1 is a diagram showing a network having a plurality of nodes and an initial configuration of active nodes and inactive nodes;

FIG. 2 is a diagram showing the network of FIG. 1 and the plurality of nodes having an intermediate configuration of active nodes and inactive nodes;

FIG. 3 is a diagram showing the network of FIG. 1 and the plurality of nodes having a final configuration of active nodes and inactive nodes;

FIG. 4 is a flowchart depicting an overall method for steering node expression towards a target attractor state; and

FIG. 5 is a diagram showing how different environmental pressure in both space and time selects cells undergoing mutation events that favor the emergence of newly intended cell types;

FIG. 6A-6D are a series of diagrams showing duplication and mutation events leading from cell type 1 to 2, and 3, and from cell type 2 back to 1;

FIGS. 7A-7C are diagrams showing a duplication event and a chromosome loss event of phenotype ϕ defined by the expression of the two genes represented by dark gray boxes, and the non expression of the two genes shown in light gray tone;

FIGS. 8A and 8B are diagrams showing the phenotype of FIGS. 7A-7C with arrows showing the regulation induced by an ideally connected gene which activates exactly the genes that are supposedly expressed;

FIG. 9 is a block diagram illustrating an exemplary computing system for implementation of a system for steering node expression towards a target attractor state; and

FIG. 10 is an illustration showing phenotypic change induced by the controlled expression of genes 4-7 of the system of FIG. 1.

Corresponding reference characters indicate corresponding elements among the view of the drawings. The headings used in the figures do not limit the scope of the claims.

### DETAILED DESCRIPTION

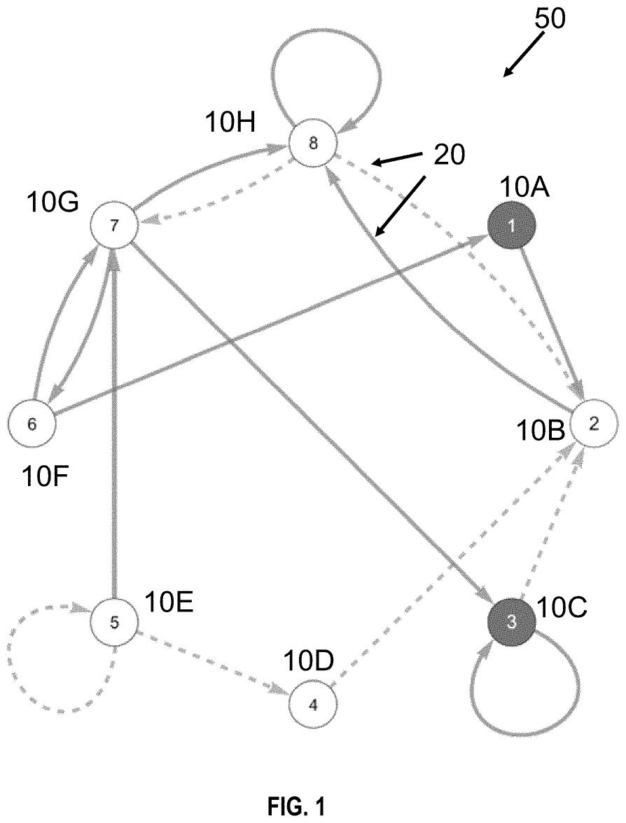

Various embodiments of a system and associated method for steering node expression towards a target attractor state in complex networks are disclosed herein. The present system identifies existing components of a complex network, and then duplicates them in a specified order to engineer a new network whose dynamics converge on the desired attractor—without requiring external intervention. In particular, the system identifies existing nodes in a network having a plurality of nodes which can be duplicated within the network to steer the network dynamics to a desired attractor state configuration. In one aspect, the complex network is a gene regulatory network and each node is a gene. Referring to the figures, a system and method for steering node expression in a complex network are illustrated and generally indicated as 100 and 200 in FIGS. 1-10.

Existing methods for network steering implement interventional approaches, where a network must continually be perturbed by altering the state of individual nodes in order to reach a target state for the system. In these methods, the absence of intervention means the absence of the target state being achieved, meaning the attractor state is not a robust feature of the dynamics but requires the continuous intervention of an engineer. The present method has the advantage that one can engineer a new behavior into a network, making the attainment of the specified attractor a robust feature of the dynamics of the network. The present method is therefore more efficient because one does not need to continually intervene. It also generates robust networks, using recycled parts (components present in the original network) so does not require design overhead in making new components from scratch. An additional feature and benefit is that it provides an open-ended solution: any attractor can be engineered into the network by the present method by a sufficient number of duplication steps, which can be set out by the present method.

The invention is a method for steering the dynamics of a network by duplicating existing nodes within the given network. A general problem in network engineering is determining the most efficient mechanism for steering a network to attain a desired attractor state. For example, in genomic networks, attractors correspond to cell types and the challenge is what interventions to apply to steer the entire network to the state it acquires within a given cell type. Likewise, one might view communications network from a similar perspective and ask how the global dynamics of a network can be steered by a specific sequence of controlled interventions. With the present invention one can take a network with known dynamics and identify the nodes whose duplication and/or mutation can steer the network's dynamics towards any attractor state specified by the engineer. This can be done without requiring that the desired attractor state already exists within the dynamics of the original network, meaning the design of what attractors the system can be steered to is open-ended and up to the engineer. Once a target attractor is identified, the present method determines the steps necessary, via duplication events, to steer to that attractor through selective modification of the original network. It is more robust than other methods because the nodes driving the network to the specified attractor are engineered into the final network, rather than requiring external interventions as in previous approaches. This feature also makes the technology developed based on the present method more efficient since no external intervention is required. A third feature is that the present method permits engineering novel phenotypes by duplicating currently existing components, meaning novelty can be engineered into the behavior of a network without require novelty in its component parts. Applications include the modification of biological networks in place of drug therapy, steering collective behavior, agricultural applications to improve crop or livestock yield, identification of changes to electrical grids to both optimize energy flow and improve fail-safes, and design of computer networks to improve stability, security, and robustness.

A critical problem in the engineering of complex networks is the capacity to steer systems to a specified attractor state. Current methods can do this, but only by continual intervention on the dynamics of a system, which is energetic costly and means that a complex network cannot achieve the desired state in the absence of the intervention. Additionally, if intervention is terminated, the network has no intrinsic capacity to regulate its own dynamics to converge to the attractor of interest. The present method solves this problem by utilizing existing components of a complex network, and then duplicating them in a specified order to engineer a new network whose dynamics converge on the desired attractor—without requiring any external intervention.

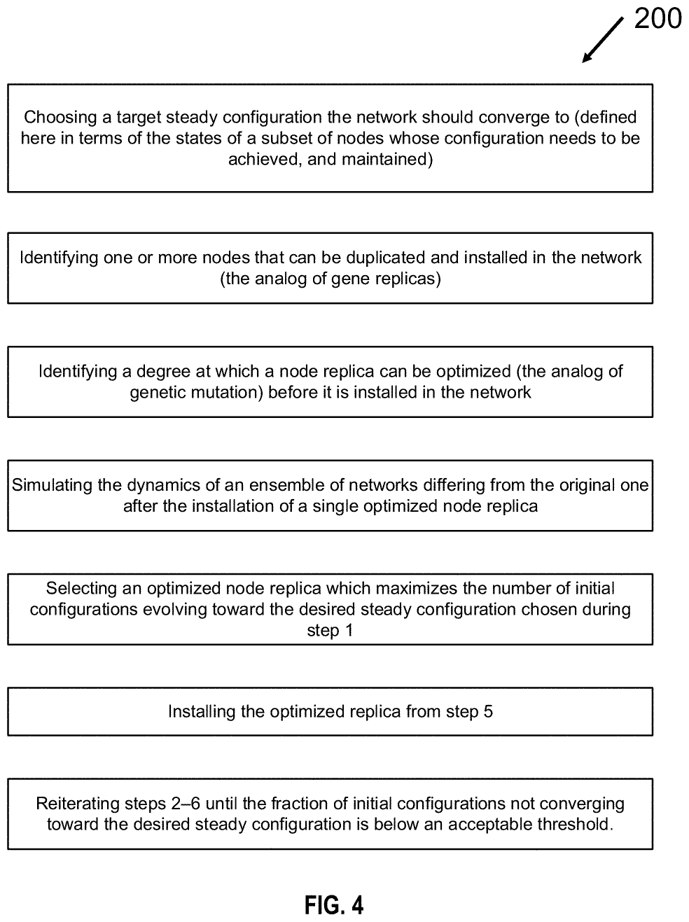

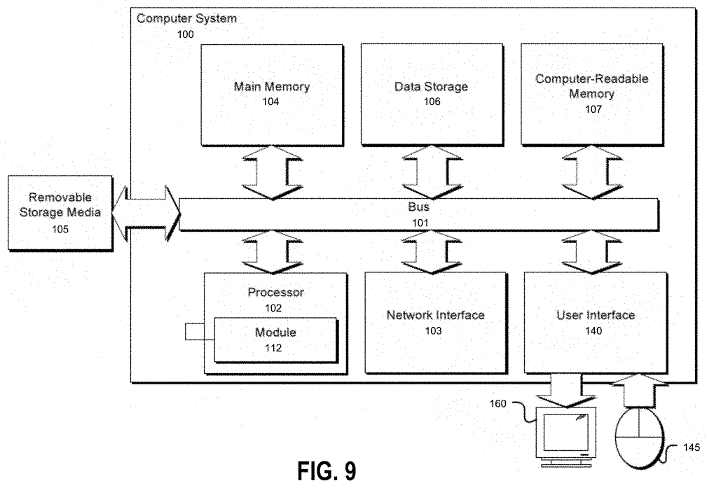

The inception of the present system 100 (FIG. 9, running method 200 of FIG. 4) came from asking a very different question—how do new cell types emerge through the process of gene regulatory network evolution? It was found that gene duplication events can allow one to explicitly determine the sequence of states to ‘evolve’ a gene network whose main attractor is a one cell type into a network whose main attractor is that of one's choosing. This observation is the foundation of the system 100 in this disclosure. Inspired by biological design, the broad utility of the system 100 was recognized for engineering specific attractors into complex networks by recycling components that already exist (as opposed to needing to invent or otherwise generate new ones). The system 100 therefore increases robustness through redundancy, meaning the target attractor state become a permanent part of the network's dynamics—even in the absence of intervention, which also saves resources by not having to continually steer the system 100.

The system 100 takes inspiration from structural changes emerging in the networks of interactions describing genetic regulatory processes. After successive phases of gene duplication, mutation and natural selection, new network structures are selected that enhance the fitness of evolved species.

### System and Method Overview

Referring to FIG. 1, gene regulatory processes can readily be modeled by Boolean networks. In a Boolean network, each node at a given time t is in one of two possible states: 0 or 1, ON or OFF, up or down, etc. In the case of a gene regulatory network, the two states correspond to a state of a gene the node represents to either be expressed or inhibited. In FIG. 1, a plurality of nodes 10A-10H of a network 50 are shown being either active (blue) or inactive (gray). The system 100 will work under the assumption that the states of each node 10 of the network 50 can be well described by Boolean variables. The interpretation of these binary states will depend on the specific nature of the network 50. It important to stress that the viability of the control method does not rely on the number of possible states as long as the following conditions are satisfied: a) The network can only be in a finite number of possible configurations; and b) Transition among these configurations are deterministic.

At a given time t, the configuration of the network is known when the state xᵢ(t)=0, 1 of every node i is known. Therefore, if the network describes the interactions among n nodes, its configuration at time t is fully specified by an n-dimensional boolean array x₁(t), . . . , xₙ(t). The dynamics of the network is then described by n boolean functions ƒ₁, . . . , ƒₙ which provide the configuration of the network at time t+1, given its configuration at time t:

x₁(t+1)=ƒ₁(x₁(t), . . . ,xₙ(t))

. . .

xₙ(t+1)=ƒₙ(x₁(t), . . . ,xₙ(t))

For n nodes, 2ⁿ possible configurations exist, and the dynamics of the network is represented by a trajectory (a time series) in the discrete space containing the totality of these configurations. For deterministic functions ƒᵢ, and because of the finite size of this configuration space, these trajectories will eventually converge to either a fixed configuration or a cycle of configurations. These special activation patterns take the name of attractors of the Boolean network, and represent the steady configurations following a transient time. Controlling a network means determining beforehand the steady configurations it will achieve after the transient time.

Nothing has been said so far about the functional form of the Boolean functions ƒᵢ, because they depend on the specific nature of the network. But for the sake of clarity, and in order to build toy models aimed at showing how the present method works, the case of Boolean network with thresholds is referred to:

$${x}_{i} ( t + 1 ) = sgn ( {\sum }_{j = 1}^{n} {a}_{ji} {x}_{j} ( t ) - {b}_{i} ) ,$$

where aⱼᵢ is the relative weight of the regulatory signal from node j to node i (activation when aⱼᵢ is positive, inhibition otherwise), and bᵢ is the activation threshold of node i.

Now that the groundwork for the numerical models has been discussed, attention is directed back to the interpretation of the attractors of a Boolean network as its steady configurations. In the biological case inspiring this proposal, the emergence of new cell types has a precise mathematical meaning as the acquisition of new attractors of a gene regulatory network, which can arise due to mutations in network structure (e.g. changes in the genotype due to gene duplication or whole genome duplication).

In the following, a method for controlling an abstract network by mimicking this natural process is shown. While standard theory of network control consists in forcing the state of a minimal set nodes sufficient to steer the dynamics of the entire network, the system 100 introduces structural changes which do not require continuous exertion of control over the network. While the traditional approach, when applied to gene regulatory networks, constitutes the theoretical basis for drug treatments, the present approach is aimed at genetically reverse-engineering the cell.

A flowchart depicting a method 200 executed by the system 100 within a network is shown in FIG. 4. The method 200 includes the following steps:
1. Choosing a target steady configuration the network should converge to (defined here in terms of the states of a subset of nodes whose configuration needs to be achieved, and maintained);
2. Identifying one or more nodes that can be duplicated and installed in the network (the analog of gene replicas);
3. Identifying a degree at which a node replica can be optimized (the analog of genetic mutation) before it is installed in the network;
4. Simulating the dynamics of an ensemble of networks differing from the original one after the installation of a single optimized node replica.
5. Selecting an optimized node replica which maximizes the number of initial configurations evolving toward the desired steady configuration chosen during step 1;
6. Installing the optimized replica from step 5;
7. Reiterating steps 2-6 until the fraction of initial configurations not converging toward the desired steady configuration is below an acceptable threshold.

The following example illustrated in FIGS. 1-3 is aimed at clarifying the way the present system 100 works in practice.

Steps 1 and 2: Consider an 8-node network 50 (FIG. 1) including nodes 10A-10H, in which node 10A, 10B and 10C represent a subset of nodes whose configuration which are to be assigned, while nodes 10E through 10H are the nodes which can be duplicated and installed in the network. Links 20 between the nodes are illustrated in FIGS. 1-3 using continuous or dashed lines; continuous lines denoting a relationship wherein a node activates another node and dashed lines denoting a relationship wherein a node inhibits another node. In the example shown in FIG. 1, a Boolean threshold network is used with equal weight edges aⱼᵢ=±1 for activating or inhibiting links. Non-zero thresholds b₁=−1 and b_{5,6,8}=1. In the example shown in FIG. 1, the steady state configuration for more than 80% of the possible initial configuration is characterized by the pattern x₁=1, x₂=0, and x₃=1, which will be referred to as Configuration 1. In this example, the remaining initial configurations need to converge instead toward a target configuration x₁=1, x₂=1, and x₃=0. This second pattern will be referred to as Configuration 2. The aim of the system is to steer the dynamics from Configuration 1 to Configuration 2, in the sense that the system 100 warrants that at least 80% of the initial configurations of the network to converge toward a steady state satisfying Configuration 2 (i.e. 20% is the acceptable threshold of step 7). For simplicity, the system 100 steers the dynamics toward a steady state which is initially unlikely, but already encoded in the original network. But this is not a requirement. A target configuration may be of any other final pattern, x₁, x₂, x₃, and the system 100 would work in the same way in order to make the target configuration the more likely steady state configuration for the network.

Step 3: First it is necessary to provide a formal definition of what is meant by an “exact” node replica, before describing how the node replica can be optimized by introducing “mutations”. Given a Boolean network N₁ with n nodes, it is necessary to study the dynamics of the n+1-node network N₂ obtained by the inclusion of the replica of a node already present in N₁.

If the dynamics of N₁ is governed by the set of Boolean equations

x′₁=ƒ₁(x₁, . . . ,xₙ)

. . .

x′ₙ=ƒₙ(x₁, . . . ,xₙ),

then the updating rules of N₂ will have the form

x′₁=φ₁(x₁, . . . ,xₙ,xₙ₊₁)

. . .

x′ₙ=φₙ(x₁, . . . ,xₙ,xₙ₊₁)

x′ₙ₊₁=φₙ₊₁(x₁, . . . ,xₙ,xₙ₊₁)

In the previous equations, if xᵢ is the value (0 or 1) of node i at time t, then x′ᵢ is the simplified notation for the value of the same node at time t+1. The (j functions are “new” functions that should be determined, starting from the knowledge of the functions ƒᵢ. Without lacking generality, assume node n+1 to be an exact replica of node 1. Then consider the requirements imposed on φᵢ (x₁, . . . , xₙ, xₙ₊₁), after inclusion of node n+1:

a) The assertion that node n+1 is an exact replica of node 1 translates into the assumption that:

φₙ₊₁=φ₁.

Thus, node replicas obey the same updating rules as the original nodes.

b) The new node should not affect the system when not expressed. Therefore,

φᵢ(x₁, . . . ,xₙ,0)=ƒᵢ(x₁, . . . ,xₙ)(i=1, . . . ,n)

c) Lastly, nodes 2, . . . , n should see node 1 and n+1 as indistinguishable:

φᵢ(0,x₂, . . . ,xₙ,xₙ₊₁)=ƒᵢ(xₙ₊₁,x₂, . . . ,xₙ)(i=1, . . . ,n)

The previous conditions are enough to determine the values taken by the functions φᵢ for any dynamical state of N₂ with the only exception of those cases when x₁ and xₙ₊₁ are both 1. This is the new scenario, whose output cannot be predicted in terms of an equivalent configuration of N₁. The problem is easily solved in the case of Boolean networks with threshold, as the prescription that gives the updating rule of a node in terms of the state of the network is known. Building the functions φᵢ in a similar fashion, it is natural, to impose a_{j+n,i}=aⱼᵢ and bᵢ₊ₙ=bᵢ for the previous three conditions to be satisfied.

Optimization of a node replica will be further discussed. The signaling of node replica i is represented by the string of numbers aᵢⱼ=0, ±1, with j=1, . . . , n (n=current number of nodes in the network). A “mutation” is introduced by changing one of these numbers to another possible value. For example, changing aᵢⱼ from 0 to ±1 corresponds to the creation of a new edge. Changing it from ±1 to 0 means deleting an edge which is instead departing from the original node. Changing it from ±1 to ∓1 changes an activating link into an inhibiting one or vice versa. This would correspond to the case in which modification of at most one outgoing edge of node replica i is allowed. The same could be true for the incoming edges. In some scenarios, more radical mutation can be introduced and more than one edge at a time. Different models of mutation could be assumed, and they determine the minimum numbers of replicas the network needs to acquire in order to change from one type to another. The kind of optimization that are feasible will depend on the specific nature of the network, be it a gene regulatory network or a different type of complex network. In the example of FIGS. 1-3, it is assumed that at most one outgoing edge can be altered at a time.

Step 4: For each possible optimized node replica, new network dynamics are simulated, using the dynamical rules described previously devoted to step 3. For each one of these networks, the percentage of initial states converging toward the chosen configuration that the system is trying to reinforce is counted.

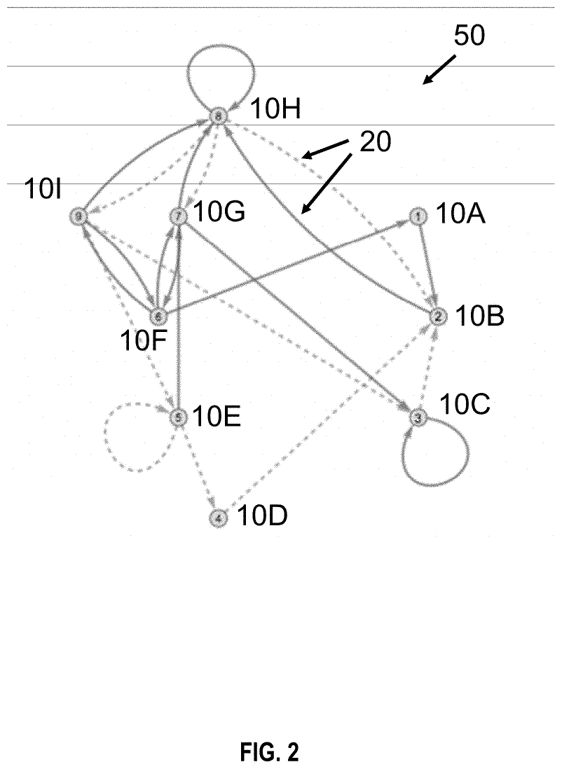

Step 5: The optimized replica providing the largest boost to the likelihood of the target configuration which is being enforced is selected. In the example of FIGS. 1-3, it is necessary to reinforce configuration 2 (see FIG. 2), and the largest improvement in the percentage of initial configurations evolving toward it would be provided by the installation of a modified replica node 101 of node 10G which is mutated to suppress node 10C instead of activating it. FIG. 2 in particular illustrates reinforcement of configuration 2, characterized by active nodes 10A and 10B and inactive node 10C. The installation of an optimized replica of node 10G that suppresses node 10C instead of activating it makes steady configurations 1 and 2 equally likely.

Step 6: The modified node replica 101 so identified is installed into the network. Steady configurations 1 and 2 are now equally likely. As 50% of the initial configurations is above the margin of error (20%), the system 100 will proceed to the next step.

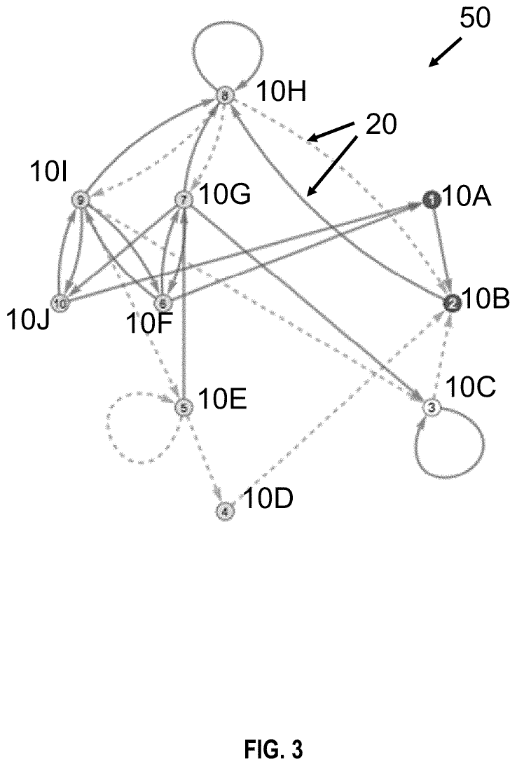

Step 7: The previous procedure is iteratively repeated until 80% of the initial configurations of the network converge to configuration two. In the example of FIGS. 2-3, the installation of a single additional replica 10J is enough: an optimized replica of node 10F that does not activate node 10G is enough to make the likelihood of configuration 2 as high as 88%. The final network in shown in FIG. 3, which illustrates the second phase of reinforcement of configuration 2. Installation of an optimized replica of node 10F that does not activate node 10G allows configuration 2 to represent a dominant steady state (88%).

The present system 100 can engineer a new behavior into a network, making the attainment of the target attractor configuration a robust feature of the dynamics of the network. The system 100 does not require continual intervention. The system 100 also generates robust networks, using recycled parts (components present in the original network) so does not require design overhead in making new components from scratch. An additional feature and benefit is that the system 100 provides an open-ended solution: any attractor configuration can be engineered into the network by the system 100 by a sufficient number of duplication steps, which can be set out by the system 100.

The advantage of this methodology is it takes advantage of network structure to ensure control, rather than relying on an external control circuit that must exert some sort of influence on the network and monitor feedback to ensure the desired dynamics. This makes it much easier to engineer robustness into systems. It also facilitates failure analysis and relates network attractor states to each other and to the network structure. This is broadly applicable across multiple systems

In addition to pre-designing control through network structure, the system 100 allows the determination of the relationships between the attractors of an original network and a recovered network with duplications. For example, the method 200 implemented by the system 100 can be applied to guide the treatment of a patient in a genetics clinic who has a multi-gene duplication where it is unclear exactly how the duplication leads to the observed phenotype. Currently, treatment becomes a series of trials and errors in attempt to recover normal function. With this method 200 implemented by the system 100, treatment can be targeted with a better chance of success. In the context of duplication with mutation, this method will identify both the node(s) to duplicate and the type of mutations to make in the duplicated node(s) to arrive at the desired attractor. This has broad application in the field of genetic engineering, particularly for GMOs. For example, one could envision identifying the endogenous gene that needs to be duplicated and mutated in order to confer increased pest resistance, eliminating the need to involve horizontal gene transfer and therefore mitigate the public's perception of GMO risk.

### INDUSTRIAL APPLICATIONS

Pre-designing network control: The method 200 allows control to be engineered into the structure of the network, essentially making the network structure the control module. This is in contrast to current methods which rely on imposing control through an outside control module that is distinct from the network. Such modules may be engineered in Matlab and Simulink, relying on the methods therein to design and simulate the networks and their dynamics. That said, the method 200 is not limited to any one particular software platform, and an implementation could be developed for Matlab and Simulink to make it widely available, but other software implementations are contemplated. In other words, the method of claim 1 may be applied to engineering network structures for simulation models through selective duplication/mutation of network components.

Determining the relationships between attractors: This method 200 allows the determination of the relationship between the attractors of the original network and a recovered network with duplications and duplications with mutation. This is applicable in the context of precision medicine. Consider a patient in a genetics clinic who has a multi-gene duplication where it is unclear exactly how the duplication leads to the observed phenotype. Treatment becomes a series of trials and errors in attempt to recover normal function. This method 200 can compare the gene network and its dynamics as it exists in the patient and relate it to the dynamics of the healthy network, identifying where therapy can best be applied to restore normal function. In the context of duplication with mutation, this method 200 will identify both the node(s) to duplicate and the type of mutations to make in the duplicated node(s) to arrive at the desired attractor. This has broad application in the field of genetic engineering, particularly for GMOs. For example, one could envision identifying the endogenous gene that needs to be duplicated and mutated in order to confer increased pest resistance, eliminating the need to involve horizontal gene transfer and therefore mitigate the public's perception of GMO risk. Thus, the method of claim 1 may be applied to determining a relationship between the attractors of an original network and a recovered network through selective mutation/duplication of cells/genes for precision medicine applications.

Synthetic cells: The method 200 would be particularly useful in designing synthetic cells optimized for the production of specific proteins. For example, the method of claim 1 may be applied to designing synthetic cells which produce specific proteins through selective cell/gene mutation/duplication.

Designing novel metabolic networks to produce useful natural products: When designing metabolic networks where there are multiple intermediates, it can be difficult to tune the network such that the reactions produce enough of the finished product because intermediates have unintended effects on the system. This method could be applied to determine how such a network, operating in a given biological background, could be optimized and/or the background modified to drive production of the final product through the duplication or targeted mutation of specific node(s). The method of claim 1 may be applied to optimizing metabolic networks to produce improved natural products for use in manufacturing through selective cell/node/gene mutation/duplication.

Controlling tumor evolution: This method when applied to a synthetic lethality paradigm in cancer treatment could allow doctors to design therapeutic regimens that select for benign growth patterns through a process where the only cells that can survive the treatment are those that duplicate and/or mutate specific genes. So for example, the method of claim 1 may be applied to a synthetic lethality paradigm to design a therapeutic regimens through selective cell/node mutation/duplication.

Increasing reliability of manufacturing processes: In manufacturing, being able to model and predict the rate and therefore amount of finished product from raw materials in a fundamental task that relies on modeling material flow through a production line. If this process can be modeled as a network where nodes represent equipment and the attractor states represent final production rates, then this method can identify those nodes that need to be duplicated (e.g. which machines and/or processes need more capacity) to maintain production rate and units at a desired level. The method of claim 1 may be applied to modeling manufacturing processes through selective equipment/node duplication/modification to improve production line quality and yield.

Optimizing node redundancy to increase the robustness of the network to perturbation: For networks where engineering is constrained by the economics of developing novel nodes or creating entirely new networks, this method can identify the node that need to be duplicated and/or modified to maintain current network dynamics even if other nodes are lost or otherwise changed. For example, consider a power grid that needs to be modernized to improve reliability. This method can identify which components of that grid need to be duplicated in order to maintain reliable service even in the event other parts of the network fail. The method of claim 1 may be applied to modeling networks such as power grids, communication networks (i.e. cellular and internet networks) through selective duplication/modification of network components to maintain reliable service and prevent overload of the system even if other components of the network fail.

### Further Discussion and Examples

One aspect of the present disclosure encompasses a method for steering the dynamics of a network. The method includes the steps of: (a) mapping the network to identify nodes in the network; (b) choosing a desired steady configuration for the network to converge to; (c) identifying nodes in the network that can be duplicated and installed in the network; (d) identifying the degree at which a node replica can be optimized; (e) numerically simulating the dynamics of an ensemble of networks differing from the original network after the installation of a single optimized node replica; (f) selecting the optimized node replica which maximizes the number of initial configurations evolving toward the desired steady configuration chosen during step b; (g) installing the selected optimized replica of step f; and (h) repeating steps c-g until the fraction of initial configurations not converging toward the desired steady configuration is below an acceptable threshold. The desired steady configuration can be the states of a subset of nodes whose configuration needs to be achieved and maintained. Further, the threshold can be 20%.

In some aspects, the abstract network is a biological network. The biological network can be a gene regulatory network. When the biological network can be a gene regulatory network, the nodes that can be duplicated and installed in the network are analogous to gene replicas. The degree at which a node replica can be optimized can be a genetic mutation. The biological network can be selected from a metabolic network or a gene expression network associated with cancer growth and development.

The abstract network can also be a manufacturing process or an engineered network.

Another aspect of the present disclosure encompasses a method for steering a gene regulatory network towards a desired outcome. The method includes the steps of: (a) choosing a desired outcome for the network to converge to; (b) identifying genes in the regulatory network; (c) identifying genes in the network that can be duplicated and installed in the network; (d) identifying the degree at which a gene replica can be optimized; (e) numerically simulating the dynamics of the ensemble of networks differing from the original network after the installation of a single optimized gene replica; (f) selecting the optimized gene replica which maximizes the number of initial configurations evolving toward the desired outcome of the gene regulatory network; (g) installing the selected optimized replica of step f; and (h) repeating steps b-g until the fraction of initial configurations not converging toward the desired outcome of the gene regulatory network is below an acceptable threshold. The desired outcome can be an improvement in the production of a metabolic product of the gene regulatory network. The desired outcome can also be control of tumor evolution.

Yet another aspect of the present disclosure encompasses a method for designing a novel metabolic network to produce a useful product. The method includes the steps of: (a) choosing a desired degree of improvement in the production of the product to converge to; (b) identifying genes in a metabolic network capable of producing the natural product; (c) identifying genes in the network that can be duplicated and installed in the network; (d) identifying the degree at which a gene replica can be optimized; (e) numerically simulating the dynamics of the ensemble of networks differing from the original network after the installation of a single optimized gene replica; (f) selecting the optimized gene replica which maximizes the number of initial configurations evolving toward production of the product; (g) installing the selected optimized replica of step e; and (h) repeating steps b-f until the fraction of initial configurations not converging toward the production of the product is below an acceptable threshold.

One aspect of the present disclosure encompasses a method for controlling tumor evolution. The method includes the steps of: (a) choosing a desired growth pattern of the tumor indicative of benign growth of the tumor to converge to; (b) identifying genes in a gene expression network associated with growth and development of a cancer; (c) identifying genes in the network that can be duplicated and installed in the network; (d) identifying the degree at which a gene replica can be optimized; (e) numerically simulating the dynamics of the ensemble of networks differing from the original network after the installation of a single optimized gene replica; (f) selecting the optimized gene replica which maximizes the number of initial configurations evolving toward benign growth; (g) installing the selected optimized gene replica of step e; and (h) repeating steps b-f until the fraction of initial configurations not converging toward benign growth of the tumor is below an acceptable threshold.

An additional aspect of the present disclosure encompasses a method for increasing reliability of a manufacturing process. The method includes the steps of: (a) choosing a desired level of reliability for the process to converge to; (b) identifying steps in the manufacturing process that can be duplicated and installed in the network; (c) identifying the degree at which a replica of a step can be optimized; (d) numerically simulating the dynamics of the ensemble of networks differing from the original network after the installation of a single optimized replica of a manufacturing step; (e) selecting the optimized step replica which maximizes the number of initial configurations evolving toward increased reliability of the manufacturing process; (f) installing the selected optimized replica of step e; and (g) repeating steps b-f until the fraction of initial configurations not converging toward increased reliability of the manufacturing process is below an acceptable threshold.

One aspect of the present disclosure encompasses a method for improving the reliability of an engineered network. The method includes the steps of: (a) choosing a desired degree of improvement in the reliability of the engineered network to converge to; (b) identifying components in the engineered network that can be duplicated and installed in the network; (c) identifying the degree at which a component replica can be optimized; (d) numerically simulating the dynamics of the ensemble of networks differing from the original network after the installation of a single optimized component replica; (e) selecting the optimized component replica which maximizes the number of initial configurations evolving toward improving the reliability of the network; (f) installing the selected optimized replica of step e; and (g) repeating steps b-f until the fraction of initial configurations not converging toward improving reliability is below an acceptable threshold. The engineered network can be a power grid.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described metabolites and methods without departing from the scope of the invention, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of”. Specifically, the term “consisting essentially of” limits membership to the specified materials or steps and those that do not materially affect the essential characteristics of the claimed invention.

### Examples

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Introduction to Examples 1-6

Abstract. The two most fundamental processes describing change in biology, development and evolution, occur over drastically different timescales. Development involves temporal sequences of cell states controlled by hierarchies of regulatory structures. It occurs over the lifetime of a single individual, and is associated with gene expression level change of a given genotype. Evolution, by contrast entails genotypic change through mutation, the acquisition/loss of genes and changes in the network topology of interactions among genes. It involves the emergence of new, environmentally selected phenotypes over the lifetimes of many individuals. One can start by reviewing the most limiting aspects of the theoretical modeling of gene regulatory networks (GRNs) which prevent the study of both timescales in a common, mathematical language. The simple framework of Boolean network models of GRNs are then considered, and its inadequacy to include evolutionary processes is pointed out. As opposed to one-to-one maps to specific attractors, a many-to-one map is adopted which makes each phenotype correspond to multiple attractors. This definition no longer requires a fixed size for the genotype, and opens the possibility for modeling the phenotypic change of a genotype, which is itself changing over evolutionary timescales. At the same time, it is shown that this generalized framework does not interfere with established numerical techniques for the identification of the kernel of controlling nodes responsible for cell differentiation through external signals.

Introduction. The Examples below are structured as follows: The next section contains a brief review of dynamical Boolean models of GRNs sufficient to orient those not familiar with the general framework. It reviews the main features of Kauffman's seminal theory, i.e. the assumption that the attractors of the network dynamics encode different, stable cellular phenotypes, permitting a model for how multiple cell type identities/fates can be encoded in the same regulatory structure. Some early advancements in the field of in silico evolution experiments of GRNs are briefly reviewed, along with the analogous reinterpretations of the genotype-phenotype mappings they required.

After a brief review of previous methods to relax the restrictive one-to-one mapping between network attractors and cell phenotypes, the redefinition of phenotypes is adopted as collections of gene expression patterns with a given subset of genes sharing the same pattern. While the traditional definition, assuming a one-to-one map between phenotype and genotype, yields increasingly fine-tuned specifications for the phenotype for progressively larger genotypes, this definition identifies the phenotypes with a macrostate, as opposed to individual (micro)states, of a dynamical system, and as such does not require fine-tuning.

The section is concluded by drawing an example network that will be used as an idealized model to explore the consequences of this generalized framework for evolutionary biology. One can now account for mutation events even when they alter the size of the genome, by the inclusion of new genetic material or deletions, and describe an idealized evolutionary model.

Mutations like gene duplication, deletion, and divergence can be at least as impactful on adaptive evolution as changes in allele types. This disclosure focuses on the case study of gene duplication, and use it as an example of genotype-changing evolutionary process. Gene duplication has occurred in all three domains of life, and is an ancient mechanism dating to before the last universal common ancestor of all life on Earth. Most genomic evolutionary processes include at least some gene duplications, and at least 50% of genes in prokaryotes and over 90% of those in eukaryotes are the result of gene duplication. Nonetheless, with the exception of a few papers, it has rarely been discussed, at least in computational modeling, as a mechanism for evolving new regulatory patterns for cell type identity.

It will be shown that, under the relaxed assumption of identifying phenotypes as macrostates of the underlying Boolean GRN, a fixed genotypic size is not necessary for specifying or retaining phenotypes through evolutionary processes. This possibility will be exploited to study the emergence of new cell types, as well as the consolidation or loss of old types, as a consequence of the changing size and topology of the GRN over evolutionary timescales, and of shifting environmental conditions. As such, this refined model also addresses an inconsistency arising from considering concepts belonging to different levels of description of a well conceived ontology of biological objects as being modeled as same-level processes: for example, gene expression levels and phenotypes, as they were interchangeable. This approach will instead assume gene expression to be at a lower level of the ontology than phenotype, while phenotype and environment will belong to the same, higher ontological level.

The remainder of the disclosure is devoted to the aforementioned role played by extracellular signaling in development, and how this can be described mathematically through its action on a kernel of controlling nodes, defined in as the minimum number of genes/nodes whose expression it is necessary to steer the dynamics of the rest of the network toward a desired attractor (phenotype). Focusing on the developmental timescale it is shown that it is still possible to easily rephrase, generalize, and adapt the notion of control kernel within the new framework. The same idealized model is used to illustrate key concepts, and show extracellular control is now more easily achieved than in Kauffman's original framework. Therefore, such refined Boolean network model, while being suitable to studies of GRN mutations over evolutionary timescales, is still able to accommodate developmental change.

### Example 1: Boolean Models

This section describes the mathematical details of the Boolean, dynamical model that is assumed in the remainder of this disclosure. In living tissues, the intrinsic patterns of gene expression coupled with signaling input dictates cell fate. Both of these processes can readily be modeled by a Boolean network. Boolean networks were originally proposed by Kauffman as a viable mathematical model of GRNs. They permit exploration of the complex steady-state dynamics of GRNs, where the attractors of the dynamics can be identified with different cell types/fates. In this disclosure, the discussion is anchored to Boolean networks as they represent the simplest mathematical model exhibiting biological and systemic properties of real GRNs. By virtue of this simplicity they are particularly easy to interpret biologically.

Before proceeding any further, it is important to prevent a possible source of ambiguity induced by the fact that the terms phenotype, cell type, cell fates, etc. might be used interchangeably in the remainder of this disclosure. Despite the fact that cell behaviors (like apoptosis, proliferation, etc.) represent temporary conditions of a cell, while cell types (like epithelial, nerve, muscle cells, etc.) correspond to more permanent identities, they share the same mathematical counterpart when described within the framework of the Boolean network models. Both types and behaviors are the result of characteristic gene activation levels. Therefore, they will share the same description as attractors of the network dynamics. The present disclosure will often refer to both as phenotypes, and avoid the specification, unnecessary at the mathematical level of the discussion, of what biological features the network model is trying to describe. An interesting consequence of generalizing the phenotypic definition is that cell types and cell fates can now be compatible, opening the possibility for GRN dynamical models to simultaneously describe both the cell types and their behaviors.

### Example 2: Review of Boolean Models

At a given time t, the state of the Boolean model of a GRN is known when the state xᵢ(t) of every gene i is known. The Boolean nature of the model assumes two possible states for gene i: active, which corresponds to xᵢ(t)=1, or inactive, with xᵢ(t)=0. For a network describing the interactions among n genes, the state at time t is specified by an n-dimensional Boolean array x₁(t), . . . , xₙ(t). The dynamics of the GRN is then described by n Boolean functions ƒ₁, . . . , ƒₙ which provide the state of the network at time t+1, given its state at time t (synchronous update, but asynchronous models are also possible):

x₁(t+1)=ƒ₁(x₁(t), . . . ,xₙ(t))

. . .

xₙ(t+1)=ƒₙ(x₁(t), . . . ,xₙ(t)), (1)

For n genes, 2ⁿ possible states (gene expression patterns) exist, and the dynamics of the network is rep-resented by a trajectory (a time series) in the discrete space containing the totality of these states. For deterministic functions ƒᵢ, and because of the finite size of this state space, these trajectories will eventually converge to either a fixed state or a cycle of states. These special activation patterns take the name of attractors of the Boolean network, and were identified by Kauffman as corresponding to the stable phenotypes of the GRN.

Nothing has been said so far about the functional form of the Boolean functions ƒᵢ. For reasons that will become clearer, and given the conceptual nature of this disclosure, the example is restricted to the case of Boolean network with thresholds:

$${x}_{i} ( t + 1 ) = sgn ( {\sum }_{j = 1}^{n} {a}_{ji} {x}_{j} ( t ) - {b}_{i} ) , \qquad ( 2 )$$

where aⱼᵢ is the relative weight of the regulatory signal from gene j to gene i (activation when aⱼᵢ is positive, inhibition when negative, and no regulation when zero), bᵢ is the activation threshold of gene i, and sgn(x) is a unitary step function, defined by sgn(x)=0 if x≤0 but sgn(x)=1 if x>0. In a more compact notation, one can write where adjacency matrix A=(aᵢⱼ), the columns B=(bᵢ) and X=(xᵢ), and the sign function is now applied component-wise. A useful feature of these models is that they convey the topology of the network, implicit in the definition of generic Boolean functions ƒᵢ, in a very explicit way, with the edges of the network representing non-null entries of the adjacency matrix.

Now that the groundwork has been laid for the numerical models, returning back to the interpretation of the attractors of a Boolean network as the phenotypes of a GRN. Given this identification, robustness and evolvability of GRNs can be mapped directly to the evolutionary changes of the attractor landscape of the corresponding Boolean model. In this framework, the emergence of new phenotypes has a precise mathematical meaning as the acquisition of new attractors, which can arise due to mutations in the network structure.

Many steady-state attractors are permitted in a Boolean network, making it an ideal model for describing how the genomic information contained within a single initial fertilized egg is differentially expressed in so many distinct cell types in response to different regional specification and morphogenetic histories. In the absence of external regulation, the likelihood of a given phenotype, is quantified in terms of the number of initial configurations of the network converging on the attractor state encoding that phenotype. The higher the number of initial configurations leading to the same equilibrium dynamics, i.e. the production of a well-defined set of proteins, the more likely the cell phenotype that set of proteins represents will be. These probabilities are referred to as the basins of attraction of the possible phenotypes encoded in the GRN. The size of a basin of attraction is a purely mathematical measure, but the basins need to include the biologically feasible initial states for this measure to acquire meaning. The frame-work described herein is deterministic. But the gene activation functions in (1) could be easily complemented with a stochastic component. It is probably more reasonable to assume that any deterministic model is actually neglecting a stochastic component. This would induce state transitions that are not the result of the deterministic part of the dynamics. But, as long as the new state is within the basin of the same attractor, the system will converge again to the same equilibrium state after a short transient. Therefore, the size of the basin of a given attractor is also a measure of the stability of the attractor to possible stochastic fluctuations. In presence of stochasticity, not only the biologically meaningful initial state and the attractor need to belong to the same basin, but the basin needs to be large enough to contain the effect of stochastic transitions.

On the other hand, the effect that external regulation, in the form of extracellular signaling during development, might have in determining the expression of a specific subset of genes, is even more dramatic, as it might force the development toward cell types that were not even initially accessible to the unperturbed GRN. The minimum number of genes whose expression needs to be controlled, for the cell fate to be determined, is the control kernel associated to that cell type.

### Example 3: GRNs as Evolvable Systems

Most of the predictions derived within the framework rely on the assumption that different cell phenotypes correspond to different attractors of the GRN. While the connection between cell types and attractors is both numerically and experimentally well-motivated, their one-to-one correspondence might be an artifact due to the diminutive size of the mathematical models that were actually solvable in the not-so-distant past, when a small number of attractors were naturally identified with different phenotypes. Recent advances in computing power are finally making the study of much larger networks possible. One interesting example is the recent dynamical model developed by Fumiãand Martins for the integration of the main signaling pathways involved in cancer. The signaling among almost 100 different genes is responsible for 63 different attractors, that eventually correspond to only seven phenotypically distinct, and not necessarily incompatible phenotypes. At the top of this phenotypical hierarchy, the authors identify only three incompatible cell fates: apoptotic, quiescent, and proliferative. An important observation is that these phenotypes are determined by just a small subset of values, e.g. the constant activation of the effector caspases in apoptotic cells, within a much larger gene activation pattern.

A further example of several attractors all sharing the same phenotypic identity is already present in the much smaller GRN describing cell-fate determination during Arabidopsis thaliana flower development. Four among ten possible attractors all represent the same inflorescence meristematic cell type.

In order to part from such a restrictive identification, and in better agreement with the works just cited, one must first adopt a mathematical redefinition of the cell phenotypes. As opposed to a single, steady state of the dynamics (or a cycle of states), they are now collections of states all sharing the same activation values (fixed or cycling) of only a subset of gene nodes. By this definition, an attractor can represent a phenotype by itself. But a phenotype can be compatible with multiple attractors. In the rest of this disclosure, a terminology will often be imported, common in Statistical Mechanics, which opposes the possible states of a system, referred to as microstates, to the phenomenological descriptions of the system in term of a reduced set of observable variables, i.e the macrostates. In this language, the difference between Kauffman's definition of a phenotype, and this definition, is reminiscent of the relationship between the microstates, and the macrostates of a physical system.

This redefinition simplifies the many-to-one mapping adopted by Tusscher and Hogeweg, where transcription factors are distinguished from phenotype genes, and the phenotypes are defined only in terms of the expression pattern of the latter. As all phenotype genes are still simultaneously needed in this latter definition, such mapping is one-to-one between the configuration space of a sub-network of the GRN and the phenotypic space, but many-to-one when the configuration space of the whole GRN is considered, because irrespective of the activation states of the transcription factors.

Early in silico works on GRN evolvability have been mainly focused on robustness, i.e. the possibility for the GRN to preserve specific old attractors while acquiring new ones, or on demonstrating how an initial set of attractors can be entirely preserved notwithstanding mutations acquired by the network. Some works show how the generation of new attractors, induced by network mutations, does not prevent the network from reversing back to older attractors under the effect of a shifting environmental pressure, and studies the emergence of such kind of evolvability. Both robustness and evolvability have been shown to evolve.

According to the redefinition, only attractors are often created or destroyed, as an effect of mutations in the network structures; not so often the phenotypes. Phenotypes are more often just reinforced as new attractors contribute with their basins, or weakened when a compatible attractor is destroyed.

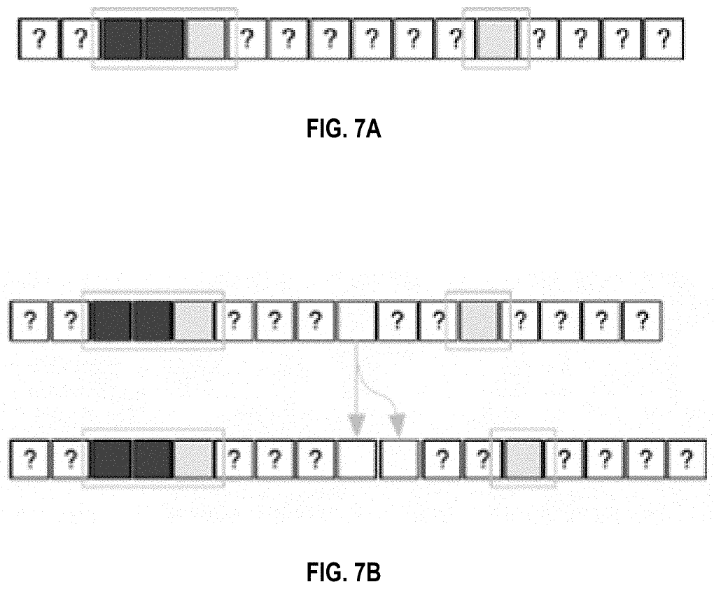

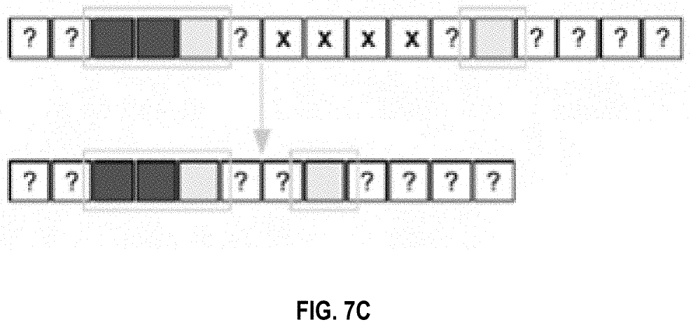

FIG. 7A will help visualize what is meant. It shows the case of a phenotype ϕ defined by the expression of the two genes represented by dark gray boxes, and the non expression of the two genes shown in light gray tone.

The expression of any other gene (boxes with a question mark) can take any value, as long as the four genes entering the definition of ϕ exhibit the right expression pattern.

A critical motivation for this redefinition of the mathematical identity of a phenotype is a conceptual difficulty which naturally arises in Kauffman's original framework. Kauffman's definition loses its strict meaning in light of evolutionary biology: the identification of the attractors of a Boolean network with the phenotypes of a GRN requires—after the regulatory network has lost or acquired genes—the comparison between genotypes of different lengths.

An immediate, advantageous consequence of this generalized definition is it keeps its meaning even after changes occur in the genotype. This is the aspect that will be explored, and the original contribution of this work. As an example, FIG. 7B shows the case of a single gene duplication event enlarging the genome expressing ϕ.

As this mutation is not affecting any of the defining genes, this change in the size of the genome does not alter this definition of ϕ. Therefore, it makes sense to study whether ϕ is still expressed by the new GRN, and whether the gene replica is favoring or disfavoring ϕ's basin. As will be further shown, these questions can be addressed in quantitative terms. The inclusion of different gene replicas will induce different changes in the relative size of the basin of attraction of ϕ.

As a second example, one can consider FIG. 7C, representing an idealized chromosome loss event. The four “x” nodes are lost, but none of them enters the definition of ϕ. Therefore, it still makes sense to ask whether the network is expressing phenotype after this deletion event.

### Example 4. Modeling Gene Duplication

In the next section, the disclosure will focus on the case study of gene duplication, and use it as an example of genotype-changing evolutionary process. Therefore, the main features of the present modeling technique are summarized herein. Let us first provide a mathematical description of what is meant by a perfect replica of a preexistent gene. Divergence in the form of a mutation affecting the replica's downstream signaling will then be introduced.

Non-mutated gene replicas: Given a Boolean network N with n nodes, it is necessary to study the dynamics of the n+1-node network N′ obtained by the inclusion of the perfect replica of a node already present in N.

If the dynamics of N is governed by the set of Boolean equations

x′₁=ƒ₁(x₁, . . . ,xₙ)

. . .

x′ₙ=ƒₙ(x₁, . . . ,xₙ),

then the updating rules of N′ will have the form

x′₁=g₁(x₁, . . . ,xₙ,xₙ₊₁)

. . .

x′ₙ=gₙ(x₁, . . . ,xₙ,xₙ₊₁)

x′ₙ₊₁=gₙ₊₁(x₁, . . . ,xₙ,xₙ₊₁)

In the previous equations, xᵢ is the value (0 or 1) of node i at time t, while is the simplified notation for the value of the same node at time t+1. The gᵢ functions are new functions that need to be determined, given knowledge of the functions ƒᵢ. Without loss of generality, one can assume node n+1 to be a perfect replica of node 1. Consider the requirements imposed on gᵢ(x₁, . . . , xₙ, xₙ₊₁), after inclusion of node n+1:
1. The assertion that node n+1 is a perfect replica of node 1 translates into the assumption that

gₙ₊₁=g₁.
Stating that node replicas obey the same updating rules as the original nodes.
2. The new node must not affect the system, when not expressed. Therefore,

gᵢ(x₁, . . . ,xₙ,0)=ƒᵢ(x₁, . . . ,xₙ)(i=1, . . . ,n)
3. Lastly, nodes 2, . . . , n need to see node 1 and n+1 as indistinguishable:

gᵢ(0,x₂, . . . ,xₙ,xₙ₊₁)=ƒᵢ(xₙ₊₁,x₂, . . . ,xₙ)(i=1, . . . ,n)

The previous conditions are enough to determine the values taken by the functions gᵢ for any dynamical state of N′ with the only exception being those cases where xᵢ and xₙ₊₁ are both 1. This is the only genuinely new scenario whose output cannot be predicted in terms of an equivalent configuration of N. The problem is easily solved in the case of Boolean networks with thresholds, as the prescription that gives the updating rule of a node in terms of the state of the network is known, eq. (2). Building the functions gᵢ in a similar fashion, it can be deduced that it is both natural, and enough, to impose a_{n+1,j}=a₁ⱼ, a_{i,n+1}=aᵢ₁ and bₙ₊₁=b₁ for the previous three conditions to be satisfied. This prescription is re-derived here, and contextualized into a more general framework of GRN described by arbitrary Boolean functions.

Mutations: The signaling of gene replica i is rep-resented by the string of numbers aᵢⱼ=0,±1, with j=1, . . . , n (n=current number of genes in the network). A mutation is introduced by changing one of these numbers to any one possible value (including its initial one, which corresponds to having a perfect replica). For example, changing aᵢⱼ from 0 to ±1 corresponds to the creation of a new edge. Changing it from ±1 to 0 means deleting an edge which is instead departing from the original node. Changing it from ±1 to ∓1 changes an activating link into an inhibiting one or vice-versa.

### Example 4: Sample Network

Mutations: In the next two sections, the consequences of the present approach in modeling evolutionary and developmental processes is explored within the Boolean model framework. To anchor the discussion to a concrete example, a small, tractable network will be adopted as an idealized model, and modified as needed.

The example is shown in FIG. 1. It represents the GRN expressing a phenotype referred to herein as cell type 1. The subset of genes defining the phenotype are represented by nodes 1, 2, and 3. The characteristic pattern of cell type 1 is assumed to have genes 1 and 3 expressed, and inactive gene 2 (blue=active, white=inactive in FIG. 1). Cell type 1 has expression pattern 101. In determining the basin of attraction of cell type 1, the basins of every attractor of the GRN which is compatible with the expression pattern 101 is summed, i.e. every pattern of the form 1, 0, 1, x₄, . . . , x₈, where x₄, . . . , x₈ (gray nodes) can take any possible (binary) value rep-resenting the expression/suppression of the remaining genes labeled from 4 to 8.

The GRN of FIG. 1 actually encodes two possible cell types, one being type 1, the other having the activation pattern 110. This second phenotype is referred to herein as cell type 2. Despite encoding both pheno-types, the basin of attraction of cell type 1 includes more than 80% of the possible initial activation patterns, all leading to the equilibrium dynamics characterized by the expression of genes 1 and 3, and the suppression of gene 2. This is why this network is assumed to be a viable description of cell type 1.

For the reader interested in reproducing the present example: The GRN dynamics is modeled using a Boolean threshold network, with equal weight edges aij=±1 for activating/inhibiting links (continuous/dashed line in FIG. 1), non-zero thresholds b1=−1 and b5,6,8=1. But it is important to remark that none of the conclusions drawn in the next sections depend on the mathematical details of the example.

### Example 5: Evolution

It has been shown how a macrostate redefinition of the phenotypes of a GRN in the framework of Boolean, dynamical models is relatively robust towards mutations changing the size of the genome. In this section, we seek to show an explicit example of how this allows to quantify how beneficial/deleterious each mutation is for a given phenotype. In this respect, the advantage of this redefinition does not become evident unless mutations affecting the size of the genotype are considered, i.e. duplications or deletions, with duplications offering a richer spectrum of possibilities. (Given the reduced size of the present idealized network, there would be only a limited number of possible deletions.) For these reasons, the present disclosure will mainly focus on duplications in what follows. But it is important to stress that any conclusions would be equally applicable to deletions and larger networks.

In the present example a chain of optimal duplications will be identified followed by divergence, until a complete shift of the network dynamics is induced from one phenotype to another. It is important to underline that gene duplication is note suggested as the main mechanism for GRN evolution. The present disclosure emphasizes the novel aspect of this refined model, as the study of mutations of this kind would not be possible without redefining the phenotypes.

It is also important to notice that mutations that preserve the genotype size could induce the same shift by themselves. As it is known that, unless restraints are imposed to the nature of the mutations that are allowed, it is always possible to find a change in the network topology for the dynamics of the network to converge to any given family of attractors. The intention in this section is instead to point the attention of the reader towards those cases that were impossible to include in traditional GRN dynamical models, and that can now be accommodated within the theory.

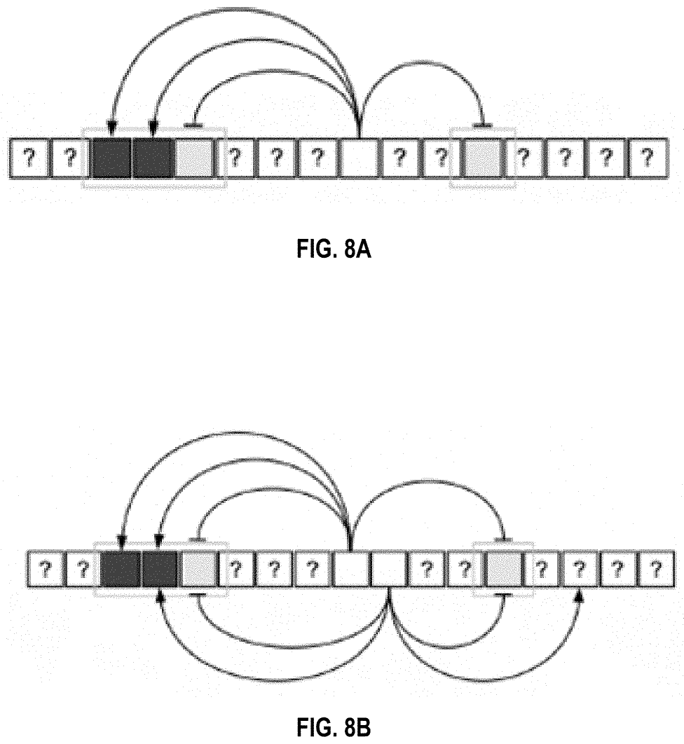

The underlying idea is exemplified in the FIG. 8A (same example of phenotype Φ adopted in FIGS. 7A-7C), with arrows showing the regulation induced by an ideally connected gene which activates exactly the genes that are supposedly expressed, and suppresses just the genes that are inactive in the former definition of Φ

It is easy to foresee that the duplication of this ideal gene would positively impact the change in the basin of attraction of ϕ.

Duplication followed by divergence is represented in the following diagram by a difference in one of the genes regulated by the replica:

In a more realistic scenario, a mutation of the new transcription factor can affect all its target genes. Alternatively, a mutation affecting the cis-regulatory regions of the target gene would affect the linkage from both the original gene, and its replica. The present simplified model is restricting the mutations only to the transcription factors. Furthermore, by mutating just one downstream connection at a time, the system is forced to operate with a more restrictive family of possible mutations events. This has two advantages. It keeps the present model, and the illustrative figures, simple enough. Any result that is proven under this more restrictive assumptions will still hold once more degrees of freedom are added to the model.

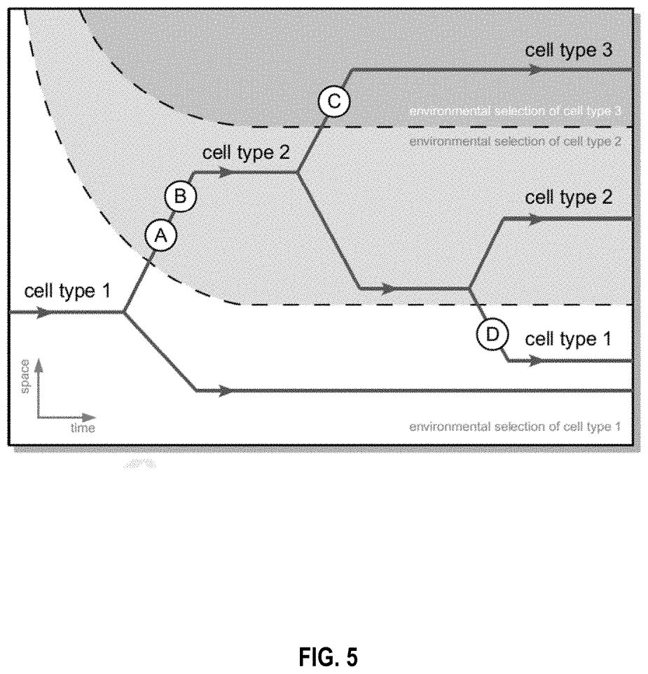

Therefore, duplication events will be considered as follows: For each gene that does not enter the definition of the phenotypes in the GRN, consider the entire spectrum of possible events of duplication+divergence of one downstream linkage, and show ideal pathways leading to cell type changes in a shifting environment. With reference to FIG. 5, a simple evolutionary model is described for the differentiation of progenitor cells of type 1, into cells of type 2 (already encoded, but unlikely), and the newly born cell type 3, a novel phenotype induced by the modifications the network is going through.

It is assumed here that the knowledge of the environment is equivalent to the knowledge of the selected cell type: they are on the same ontological level, and share the same mathematical definition in the present theoretical model. The environmental pressure changes in both space and time, and selects cells that undergo mutation events that favor the emergence of the newly intended cell type. In this example, sister cells of type 1 start being selected for cell type 2, and undergo mutation events A and B until cell type 2 represents the dominant phenotype. In the present model, the mutation events are represented by gene duplication and divergence of one of the genes not included in the definition of the phenotypes (nodes 4-8 in FIG. 1), during which the network acquires a non-mutated replica of a preexistent gene, and then a mutation in the way the replica regulates the remaining genes and itself. At least two mutation events are needed to convert sister cells of type 1 to type 2 (activation pattern: 110). These optimal mutations are derived as previously described in this section: All possible events of duplication+mutation are considered, and the one inducing the largest enhancement of the relative size of the basin of cell type 2 is selected. The network is then mutated accordingly, and this process iterated until cell type 2 is the dominant phenotype.

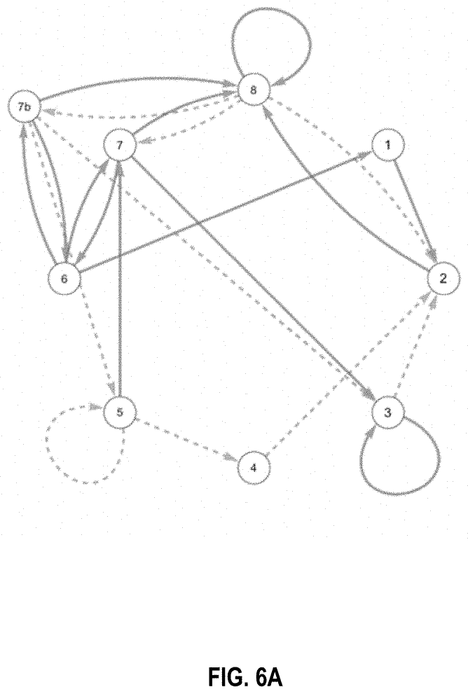

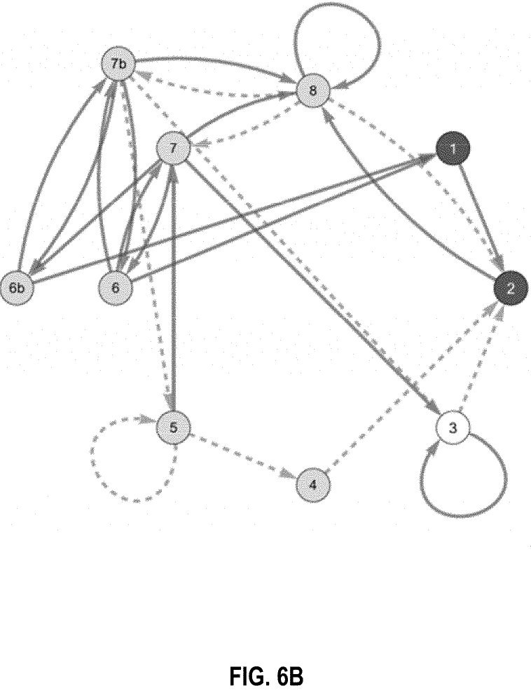

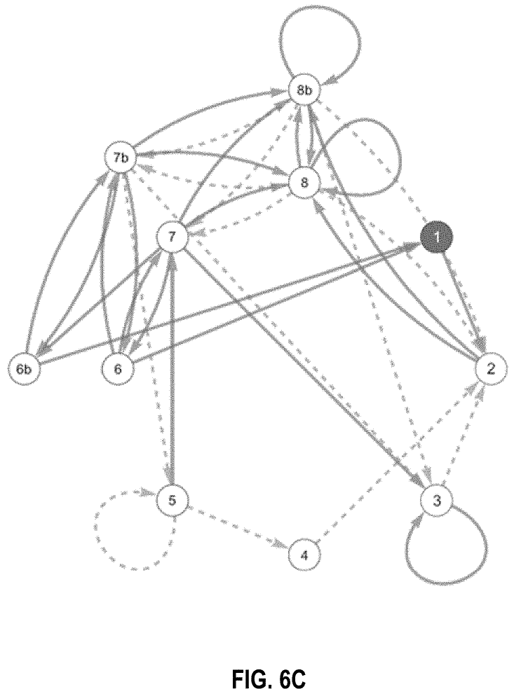

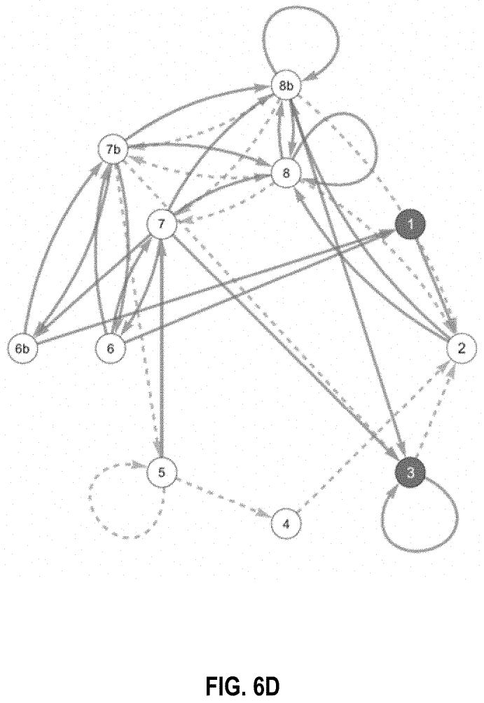

By acquiring a mutated replica of gene 7, event A, that inactivates gene 3 instead of activating it, the dynamics of the GRN is equally likely to converge toward cell type 1 or 2 (FIGS. 6A-6D). The subsequent acquisition of a mutated replica of gene 6, event B, that, differently from the original one, does not activate gene 7, determines the complete shift toward cell type 2 (88% of the configuration space). FIG. 6A in particular shows environmental selection of cell type 2, characterized by the expression of genes 1 and 2, and suppression of gene 3. Acquisition of a mutated replica of gene 7 that inactivates gene 3 instead of activating it. Mixed cell types (50% type 1, 50% type 2)._The highly connected sub-set of nodes that appear in FIG. 6B is responsible for the convergence of the dynamics toward the characteristic activation pattern which defines cell type 2. FIG. 6B shows environmental selection of cell type 2. Acquisition of a mutated replica of gene 6 that dies not activate gene 7. Type 2 cells represent now the dominant cell type.

The assumption that sister cells of cell type 2 start being selected for cell type 3 (activation pattern: 100), determines the selection of mutation events like in FIG. 6C, where the GRN acquires a mutated replica of gene 8 that suppresses gene 3. One mutation event is now enough to determine the complete shift toward cell type 3. FIG. 6C shows environmental selection of cell type 3: expression of gene 1, suppression of gene 2 and 3. Acquisition of a mutated replica of gene 8 that suppresses gene 3. Type 3 becomes the dominant cell type (92%).

The last example considered here is the convergence of cell type 2 back to cell type 1. After the acquisition of mutations A and B, the intended cell type is again type 1. This can be achieved (complete shift from less than 12% to more than 96%) with a single mutation event, shown in FIG. 6D, including a mutated replica of gene 8 that activates gene 3. This last evolution of the GRN expresses the same phenotype as the initial network in FIG. 1, but carries memory of the evolutionary path that led it through its type 2 period in the form of the modular structure visible in FIG. 6D. FIG. 6D shows, after having been selected for type 2, cells are once again selected for type 1. Acquisition of a mutated replica of gene 8 that activates gene 3. Type 1 is again the dominant cell type.

It is hoped that this example is enough to convince the reader that, as long as a viable mathematical description of the GRN is available, as well as the knowledge of the intended phenotypes for a shifting environment, the present approach can accommodate enough events of gene duplication and mutation to induce any phenotypic shift. The possibility to retain cell type mathematical identities for evolving GRNs is the key feature enabling this.

In summary, a specific family of possible mutations has been considered, in view of a rather restrictive scenario. All mutations within the chosen family have been considered, and the sequence of mutations which evolves the system toward the selected phenotype has been highlighted. This procedure was repeated for different intended phenotypes to mimic the selective pressure of a changing environment. The reader should not be surprised by the fact that convergence can always be reached, even with the few degrees of freedom allowed, as these mutations and divergence are already enough to enable open-endedness of the system.

In a population dynamics study the optimal sequences of mutations that were highlighted might be selected by some of the organisms. But, more likely, successful organisms will develop less optimal sequences leading to the same phenotype. How often this happens, and how much time it requires, ultimately depends on the choice of many parameters, like mutation rates, population size, functional form of the fitness function, etc. Given the conceptual nature of this disclosure, the main feature of the analysis should not be hidden behind the amount of parameters we would have needed to set to simulate a population. In some embodiments, no simulation was performed. Rather, a scan of all allowed mutations was performed, and the optimal ones were selected because they illustrate a point in the most synthetic way: The possibility to quantify how beneficial or deleterious each mutation is for a certain phenotype—as measured by the change it induces in the size of the corresponding basin—even in the presence of gene insertions or deletions.

It was also shown that the present method allows the identification of GRNs that differ for both genotype and network topology (like the GRN in FIG. 1 and the evolved network in FIG. 6D) as encoding the same cell type (type 1 in the example). Therefore, apparently redundant topological features in the structure of a GRN might carry the imprint of their evolutionary history, and of the environment that induced them.

A word of caution is in order regarding the computational time needed to perform the analysis. In the present example, the number of possible duplication+divergence events scales like N², where N is the current number of nodes in the network before the insertion of the new gene. Therefore, the main computational burden is still the more fundamental problem of solving the dynamics of the resulting network with N′=N+1 nodes, as it requires determining the output of equations (1) for 2^{N′} possible states. This calculation needs to be repeated for each node, or set of nodes added or removed. In the example shown, the size of the network is strictly increasing, and this induces an exponential growth of the total computational time, with each step in the calculation requiring twice the time as its previous step. In more realistic examples, the most favorable mutation might be a deletion, as opposed to a duplication. In general the main limit to the calculation will be the largest size N̅ the network achieves during the intermediate steps of the calculation. If T̅ is the computational time this network requires, then the total computational time will be less than nT̅, where n is the number or mutation events. As long as N̅≲30 the problem can be solved exactly. Beyond that threshold, approximation techniques need to be employed, as usual in this field. In the simplest possible approach, a random sampling of the configuration space might be enough to determine the size of the largest basins of attraction within an acceptable error. Otherwise, more sophisticated sampling approaches based on model checking have been reviewed recently and applied to GRN dynamics.

### Example 6. Development

Next it is necessary to show how external control of the expression of accurately chosen genes, e.g. because of extracellular signalling during development, can determine cell differentiation, and the expression of not only a subdominant phenotype, but also novel cell types that were not initially encoded in the network.

An excellent example of how extracellular signaling is included in Boolean GRN models is provided by the detailed model of spatial and temporal gene expression for embryonic development in the sea urchin. By including progressive embryonic geometry information into the equations for gene expression kinetics, the model predicts different gene expression patterns for the identical regulatory circuits within the four different spatial domains of the embryo during its first 30 hours of life. Embryonic geometry is added to the model through the specification of the relative positions of the different lineages of cells at each stage. These relative positions of the distinct domains change during development, and determine the nature of inter-domain signals. From a mathematical perspective, and within a single cell, this translates into time-dependent, external activation/inhibition signals that selectively act on subsets of genes. These external signals have the potential to set the activation states of their targets, regardless of the internal gene expression dynamics of the individual cell. Therefore, they act as a form of extracellular control.

The minimal subset of genes whose controlled expression is sufficient to induce the appearance of the new lineage takes the name of control kernel of that phenotype (see below for the rigorous, and operative definition). One goal of this disclosure is to show the role played by a generalized definition of phenotype in not only preserving the applicability of this dynamical control formalism, but also in reducing the size of the control kernel.

The original approach includes taking every possible combination of any subset of genes and their possible expression patterns. For each combination, and each initial state, time series of the transient expression pattern of the remaining genes are generated, while the selected genes are set to the specific selected values. When by setting the values of the smallest possible number of nodes this operation moves all possible initial conditions toward the basin of attraction of a specific attractor, Kim et al. defines this smallest subset of nodes as the control kernel of the attractor state the system is converging to.

For consistency with the present less constrained definition of what a phenotype is, it is necessary to redefine the notion of control kernel accordingly. The generalized definition adopted herein does not assume the forced terminal state to be a specific attractor, just a phenotype (i.e. any possible attractor compatible with the definition of that phenotype). As a result, control kernels of the present system are significantly smaller than those found in other methodologies. To show this explicitly, the sample network from FIGS. 7A-7C have been considered, and the effect of setting the value of just one among genes 4-8 at a time.

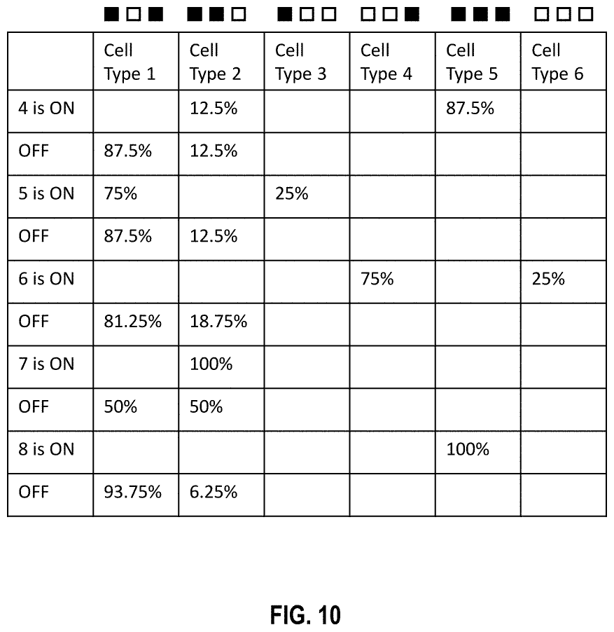

The result is shown in FIG. 10. Forcing the expression of gene 7 (referred to as “7 is ON” in the table) is enough to enlarge the basin of attraction of cell type 2 from less than 20% to exactly 100%. Likewise, forcing the expression of gene 8 makes the network shift toward a phenotype characterized by the expression pattern 111, that was not even encoded in the network. FIG. 10 illustrates phenotypic change induced by the controlled expression of genes 4-7 and shows the result of pinning just one gene at a time (TS ON/OFF). Four new phenotypes, cell types from 3 to 6, not initially encoded in the GRN of FIG. 1, are now possible equilibrium state of the gene expression dynamics, with basins of attraction sizes (likelihoods) listed as percentages.

### Summary and Future Directions.

This last section summarizes the present idea, and underlines the advantage that the present refined dynamical framework might give in describing regulatory interaction change over the evolutionary scale.

Despite their predictive power, and their possibility to mathematically reinterpret cell fates as the steady states of the abstract, non-linear dynamics of the regulatory processes they describe, dynamical models (Boolean models in this disclosure) of GRNs are often limited by this identification between mathematical attractors and biological phenotypes. Evolutionary processes, like single gene and whole genome duplication, both chromosome gain and loss, horizontal gene transfer, etc., alter the size of the genome, and deprive the exact attractor-phenotype identification as a one-to-one map of its original meaning. The leading role played in evolution and speciation by the interplay between these processes and natural selection, makes most of the current dynamical models of GRNs unfit to describe genetic network evolution.

A specific example will help us clarify what is meant. Consider the preservation, over the last half a billion years, of the complex GRN architecture responsible for endoderm specification in echinoderms, best exemplified by the similarities found in the GRNs of sea urchins and starfish. A large nucleus of interactions, preserved since the divergence of the two species from their last common ancestor at the end of the Cambrian, is surrounded by peripheral network linkages to target genes that are specific to the two species. Notwithstanding the similarities between the two networks, a theory able to quantify the effect on phenotypic change induced by the peripheral differences just described is currently missing. While a qualitative study of this change can still be addressed, a coherent mathematical framework would run into severe ambiguities, because of the difficulties in mapping arrays of different lengths—the phenotypes of the two species—into one another.

Motivated by this limitation, the consequences of relaxing this one-to-one identification between cell types and attractors have been reviewed. It was shown with a simple numerical model that, redefining a cell type as a collection of attractors of a GRN, all sharing a common, characterizing property, enables retaining the main features of Kauffman's original theory. But it is now no longer needed for the genome to retain a fixed size for the cell type definition to preserve its meaning.

Next it was important to ensure that said generalized framework did not interfere with the mathematical description of extracellular signaling in terms of its effect on a kernel of controlling genes, because how easily such technique allows to describe developmental processes in Boolean network models.

Despite its simplicity, this change might open the possibility to perform quantitative studies of the effect that genetic mutations have on cell types, by virtue of allowing the description of evolutionary processes within the well-established framework of dynamical systems theory. For this to become possible, a very detailed understanding of the regulatory structure—at least as good as Dodelson's model of the sea urchin needs to be achieved beforehand. This is probably the most severe, current limitation of the approach we are suggesting. But such models, a good understanding of the shifting selective pressure, and criteria for the identification and the mathematical description of the phenotypes across different genotypes, would open the possibility to perform in silico studies of the mutation events compatible with (if not responsible of) the observed phenotypic divergence of closely related species.

### Computing System

FIG. 9 illustrates an example of a suitable system 100 used to implement various aspects of the present system and methods for a resilient infrastructure simulation environment. Example embodiments described herein may be implemented at least in part in electronic circuitry; in computer hardware executing firmware and/or software instructions; and/or in combinations thereof. Example embodiments also may be implemented using a computer program product (e.g., a computer program tangibly or non-transitorily embodied in a machine-readable medium and including instructions for execution by, or to control the operation of, a data processing apparatus, such as, for example, one or more programmable processors or computers). A computer program may be written in any form of programming language, including compiled or interpreted languages, and may be deployed in any form, including as a stand-alone program or as a subroutine or other unit suitable for use in a computing environment. Also, a computer program can be deployed to be executed on one computer, or to be executed on multiple computers at one site or distributed across multiple sites and interconnected by a communication network.

Certain embodiments are described herein as including one or more modules 112. Such modules 112 are hardware-implemented, and thus include at least one tangible unit capable of performing certain operations and may be configured or arranged in a certain manner. For example, a hardware-implemented module 112 may comprise dedicated circuitry that is permanently configured (e.g., as a special-purpose processor, such as a field-programmable gate array (FPGA) or an application-specific integrated circuit (ASIC)) to perform certain operations. A hardware-implemented module 112 may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module 112 that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules 112 are temporarily configured (e.g., programmed), each of the hardware-implemented modules 112 need not be configured or instantiated at any one instance in time. For example, where the hardware-implemented modules 112 comprise a general-purpose processor configured using software, the general-purpose processor may be configured as respective different hardware-implemented modules 112 at different times. Software may accordingly configure a processor 102, for example, to constitute a particular hardware-implemented module at one instance of time and to constitute a different hardware-implemented module 112 at a different instance of time.

Hardware-implemented modules 112 may provide information to, and/or receive information from, other hardware-implemented modules 112. Accordingly, the described hardware-implemented modules 112 may be regarded as being communicatively coupled. Where multiple of such hardware-implemented modules 112 exist contemporaneously, communications may be achieved through signal transmission (e.g., over appropriate circuits and buses) that connect the hardware-implemented modules. In embodiments in which multiple hardware-implemented modules 112 are configured or instantiated at different times, communications between such hardware-implemented modules may be achieved, for example, through the storage and retrieval of information in memory structures to which the multiple hardware-implemented modules 112 have access. For example, one hardware-implemented module 112 may perform an operation, and may store the output of that operation in a memory device to which it is communicatively coupled. A further hardware-implemented module 112 may then, at a later time, access the memory device to retrieve and process the stored output. Hardware-implemented modules 112 may also initiate communications with input or output devices.

As illustrated, the system 100 may be a general purpose computing device, although it is contemplated that the computing system 100 may include other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the general purpose computing device may include various hardware components, such as a processor 102, a main memory 104 (e.g., a system memory), and a system bus 101 that couples various system components of the general purpose computing device to the processor 102. The system bus 101 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The system 100 may further include a variety of computer-readable media 107 that includes removable/non-removable media and volatile/nonvolatile media, but excludes transitory propagated signals. Computer-readable media 107 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the general purpose computing device. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 104 includes computer storage media in the form of volatile/nonvolatile memory such as read only memory (ROM) and random access memory (RAM). A basic input/output system (BIOS), containing the basic routines that help to transfer information between elements within the general purpose computing device (e.g., during start-up) is typically stored in ROM. RAM typically contains data and/or program modules that are immediately accessible to and/or presently being operated on by processor 102. For example, in one embodiment, data storage 106 holds an operating system, application programs, and other program modules and program data.

Data storage 106 may also include other removable/non-removable, volatile/nonvolatile computer storage media. For example, data storage 106 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules and other data for the general purpose computing device 100.

A user may enter commands and information through a user interface 140 or other input devices 145 such as a tablet, electronic digitizer, a microphone, keyboard, and/or pointing device, commonly referred to as mouse, trackball or touch pad. Other input devices 145 may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices 145 are often connected to the processor 102 through a user interface 140 that is coupled to the system bus 101, but may be connected by other interface and bus structures, such as a parallel port, game port or a universal serial bus (USB). A monitor 160 or other type of display device is also connected to the system bus 101 via user interface 140, such as a video interface. The monitor 160 may also be integrated with a touch-screen panel or the like.

The general purpose computing device may operate in a networked or cloud-computing environment using logical connections of a network interface 103 to one or more remote devices, such as a remote computer. The remote computer may be a personal computer, a server, a router, a network PC, a peer device or other common network node, and typically includes many or all of the elements described above relative to the general purpose computing device. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the general purpose computing device may be connected to a public and/or private network through the network interface 103. In such embodiments, a modem or other means for establishing communications over the network is connected to the system bus 101 via the network interface 103 or other appropriate mechanism. A wireless networking component including an interface and antenna may be coupled through a suitable device such as an access point or peer computer to a network. In a networked environment, program modules depicted relative to the general purpose computing device, or portions thereof, may be stored in the remote memory storage device.

While the present disclosure has been described with reference to various embodiments, it should be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system for steering dynamics of a network without external intervention, comprising:
data defining a network comprising a plurality of nodes and known dynamics associated with an original configuration of the network, the data further defining a desired attractor for the network associated with a predefined subset of the plurality of nodes and a predefined threshold; and
a processor having access to the data, the processor further having access to a set of executable instructions which, when executed, cause the processor to:
(i) identify a plurality of replica nodes of the plurality of nodes that can be duplicated and installed to the network,
(ii) numerically simulate new dynamics of a set of initial configurations of the network differing from the original configuration, the set of initial configurations defining subsets, wherein each of the subsets corresponds to a replica node of the plurality of replica nodes and defines initial configurations from the set of configurations with the replica node installed to the network,
(iii) select, in view of the new dynamics of the set of initial configurations, a node for installation from the plurality of replica nodes that corresponds to one of the subsets of the initial configurations with a maximum number of initial configurations evolving toward the desired attractor relative to other ones of the subsets,
(iv) install the node as selected for installation to the network, and
iteratively repeat steps (i)-(iv) until a fraction of the set of initial configurations not converging towards the desired attractor is below a predefined threshold to output a final configuration of the network.

2. The system of claim 1, wherein the original configuration of the network is modeled as a first Boolean network with n nodes expressed as an n-dimensional Boolean array and governed by a set of Boolean equations with known states for each node of the plurality of nodes.

3. The system of claim 2, wherein the dynamics of the network are represented by trajectories in discrete space containing a totality of all possible configurations of the network over a time series, wherein the trajectories, by nature of the finite size of configuration space of the network, converge to either a fixed configuration or a cycle of configurations defining attractors of the first Boolean network.

4. The system of claim 3, wherein the desired attractor for the network is an attractor of a Boolean network that represents a desired steady state configuration achieved after a transient time.

5. The system of claim 1, wherein each of the plurality of replica nodes obeys the same updating rules as corresponding original nodes of the network.

6. The system of claim 1, wherein the processor includes further executable instructions which, when executed, cause the processor to:
identify a mutation applicable to a given replica node of the plurality of replica nodes, the mutation introduced by changing a number of a string of numbers representing a signaling of the given replica node to another value to modify properties of the network.

7. The system of claim 6, wherein the mutation includes a modification to an edge of the network associated with the given replica node.

8. The system of claim 6, wherein the mutation represents an emergence of a biological sequence mutation resulting in a new cell type, and the network represents a gene regulatory network.

9. The system of claim 8, wherein the new cell type is at least one of: a predetermined cell type of a [crop] plant or a predetermined cell type of a[n] [livestock] animal, or a predetermined human cell type.

10. The system of claim 6, wherein the network is a metabolic network, and the mutation represents the emergence of a predetermined natural product of the metabolic network.

11. The system of claim 1, wherein the network is a power grid.

12. A method for steering dynamics of a network without need of external intervention, comprising:
providing a network defining a plurality of nodes in an original network configuration,
identify, by a processor, a node of the plurality of nodes of the network where duplication or mutation of the node steers dynamics of the network towards a predetermined target attractor; and
steering the dynamics of the network to the predetermined target attractor by selective modification of the original network configuration including duplicating the node and adding the node to the network.

13. The method of claim 12, wherein each of the plurality of nodes is interconnected such that activation or suppression of one of the plurality of nodes causes activation or suppression of at least one other node of the plurality of nodes.

14. The method of claim 12, wherein the network is a gene network and the target attractor is a predetermined cell type.

16. The method of 14, wherein the predetermined cell type is at least one of: a cell type of a crop plant, a cell type of a [livestock] animal, or a human cell type.

17. The method of 12, wherein the network is a power grid.

18. The method of claim 12, wherein the network is a metabolic network and the target attractor is a predetermined metabolic product.

19. A method for steering dynamics of a network, the method comprising:
mapping a network to identify one or more nodes in the network;
selecting a target attractor for the network to converge to;
identifying one or more nodes in the network that can be duplicated and installed in the network;
identifying a degree at which a node replica can be optimized, wherein the node replica is a duplication of the one or more nodes in the network that can be duplicated;
simulating dynamics of a plurality of simulated networks differing from the network after the installation of a single optimized node replica;
selecting the optimized node replica which maximizes the number of initial configurations evolving toward the target attractor; and
installing the selected optimized node replica into the network.

20. The method of claim 19, wherein the steps of identifying one or more nodes in the network that can be duplicated and installed in the network, identifying a degree at which a node replica can be optimized, simulating dynamics of a plurality of simulated networks differing from the network after the installation of a single optimized node replica, selecting the optimized node replica which maximizes the number of initial configurations evolving toward the target attractor, and installing the selected optimized node replica into the network are iteratively repeated until a fraction of initial configurations not converging toward the desired steady configuration is below a predetermined threshold.
